(19) United States
(12) Patent Application Publication
(10) Pub. No.: US 2010/0286733 A1
(43) Pub. Date: Nov. 11, 2010

(54) MENISCAL DEVICE

(75) Inventor: Scott Trenhaile, Belvidere, IL (US)

Correspondence Address: DICKE, BILLIG & CZAJA, FIFTH STREET TOWERS, 100 SOUTH FIFTH STREET, SUITE 2250, MINNEAPOLIS, MN, 55402, US

(73) Assignee: Rockford Orthopaedic Sports Medicine Services, LLC, Belvidere, IL (US)

(21) Appl. No.: 12/775,144

(22) Filed: May 6, 2010

Related U.S. Application Data
(60) Provisional application No. 61/175,998, filed on May 6, 2009.

(51) Int. Cl. A61B 17/56; A61F 2/08

(52) U.S. Cl. 606/86 R; 623/14.12

(57) ABSTRACT

A device for sizing and implanting meniscus graft tissue includes a curvilinear tray having an inner curvature edge and an outer curvature edge opposite the inner curvature edge, a top surface and a bottom surface opposite the top surface, and at least one opening formed therethrough between the top surface and the bottom surface to capture at least one element configured to releasably secure the meniscus graft tissue to the curvilinear tray; and an elongated handle extending from the curvilinear tray and including an angled shaft terminating in a handle end.

### CROSS REFERENCE TO RELATED APPLICATIONS

This application claims priority under 35 U.S.C. §119(e) to U.S. Provisional Patent Application Ser. No. 61/175,998 filed on May 6, 2009, and incorporated herein by reference.

### FIELD OF THE INVENTION

The present invention relates generally to a medical device that facilitates orthopaedic procedures requiring the delivery of material grafted to native meniscus in the human knee. The device would be utilized to properly size the defect in the native meniscus and then deliver the replacement of tissue devoid or previously removed. With this device, the appropriate size of graft may be introduced into the knee and then delivered with accuracy. The graft can then be sutured in place while being maintained in proper position, and then the device is easily removed.

### BACKGROUND

The meniscus tissue is a fibrocartilaginous structure in the knee joint which performs multiple critical functions, including contributing to normal knee bio-mechanics and the general well being of the joint. Generally, the menisci are comprised of two C-shaped fibrocartilaginous structures residing on the tibial plateau. The peripheral rim of a meniscus is thick, tapering to a thin, free inner border.

In the knee joint, the intra-articular fibrocartilage comprises the meniscus, a crescent shaped or semi-lunar shaped disk of tissue that is located between the femoral condyles and the tibial plateau. The meniscus primarily functions as a shock absorber, absorbing the shock of compressive and shear forces in the knee. The meniscus also provides a substantially frictionless surface for articulation of the knee joint.

Once cartilage tissue is no longer healthy, there can be debilitating pain in the joint. Cartilage health can be adversely affected by disease, aging or trauma. Common surgical procedures for treating meniscal damage include meniscectomies. Meniscectomies involve the surgical removal of part of the meniscus. Although meniscectomies provide immediate relief to the patient, in the long term the absence of part of the meniscus can cause cartilage wear on the condylar surface, eventually leading to arthritic conditions in the joint.

Arthroscopy of the knee is one of the most commonly performed orthopaedic procedures. Of those arthroscopies, partial menisectomy is one of the most common procedures performed. Recently, interest has evolved in the replacing of this meniscal tissue with collagen tissue to facilitate re-growth of meniscus-like tissue in its place. Arthroscopically inserting this tissue has been a challenge along with sizing of the defect being filled by the tissue graft. Current meniscal graft choices include materials that are very fragile and difficult to place and hold while being secured in place. A device that could size the meniscal defect, deliver the meniscal graft, hold the meniscal graft in place without damaging it during fixation, and be easily removed would be very beneficial. Moreover, doing all of this arthroscopy has numerous added benefits.

A desirable device provides for sizing and implantation of meniscus graft tissue in a patient for repairing a meniscus tissue in the patient.

For these reasons, there is a need for the present invention.

### BRIEF DESCRIPTION OF THE DRAWINGS

FIG. 1 illustrates a top view of one embodiment of a meniscal device.

FIGS. 2A and 2B illustrate top views of multiple embodiments of the meniscal device.

FIG. 3 illustrates a top view of one embodiment of the meniscal device.

FIG. 4 illustrates a side view of one embodiment of the meniscal device with a meniscus tissue graft attached thereto.

FIG. 5 illustrates an embodiment of the meniscal device as used for sizing of the medial meniscus.

FIG. 6 illustrates another embodiment of a meniscal device as used for sizing of the lateral meniscus.

FIG. 7 illustrates an end view of one embodiment of a meniscal device with a meniscus tissue graft attached thereto.

FIG. 8 illustrates one embodiment of a meniscal device with the meniscus tissue graft attached as inserted into the medial side of the knee.

FIG. 9 illustrates the meniscus device of FIG. 8 as prepared for removal after suturing of the meniscus tissue graft.

FIG. 10 illustrates the meniscal tissue graft of FIG. 8 after the meniscal device is removed.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

### Meniscal Device

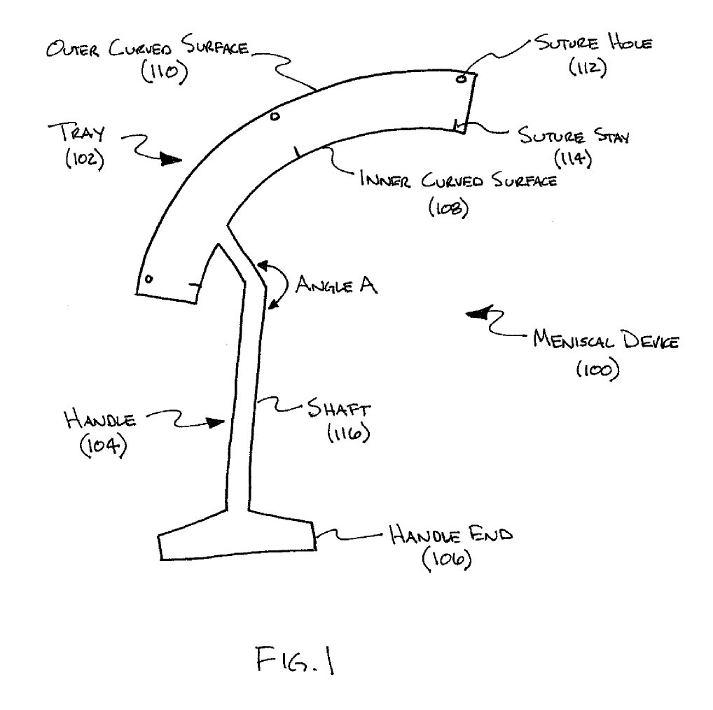

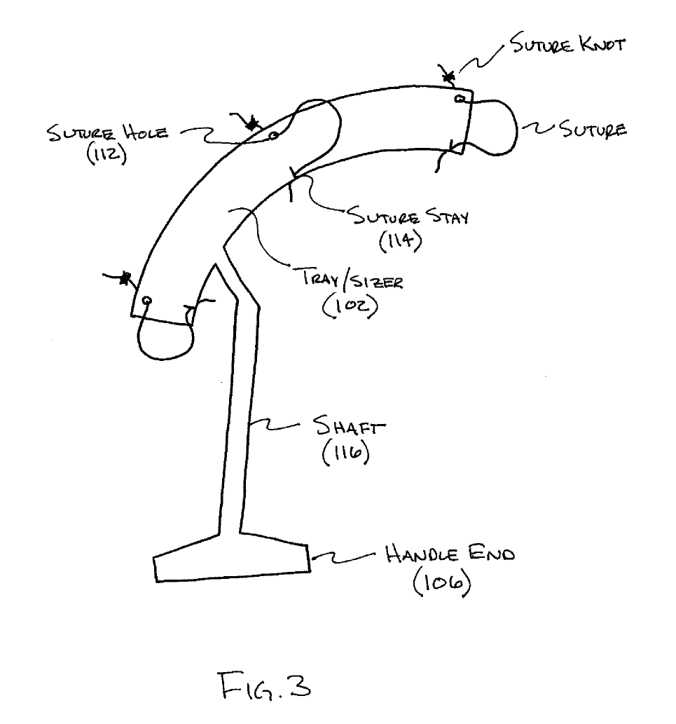

With reference to FIGS. 1-4, one embodiment of a meniscal device includes a curvilinear thin flat tray and a handle attached to the inner curvature of the tray. In one embodiment, an outer curvature of the tray includes a series of holes sized to allow absorbable sutures through and intercept the suture knot at the end of the each suture. In one embodiment, along the inner curvature of the tray, a series of slits are provided which correspond to each of suture holes provided along the outer curvature. Each slit allows the suture to pass through it, but provides an interference fit of the suture into the slit to form a suture stay. In one embodiment, a series of three suture holes and corresponding suture slits or stays are included.

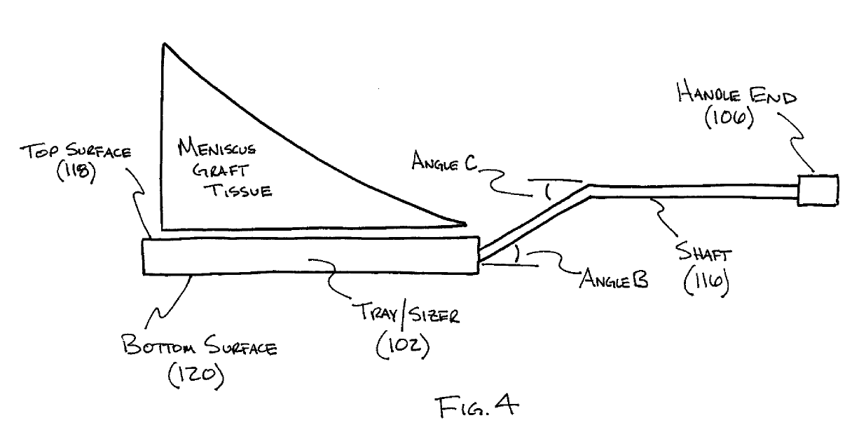

In one embodiment, one end of a handle shaft is attached to the inner curvature of the tray. In one embodiment, an opposing end of the handle shaft includes a T-shaped handle. In one embodiment, the handle shaft of the meniscal device has an angled attachment (Angle A) to the meniscal tray (FIG. 1) and another set of angles (Angle B and Angle C) that align the majority of the handle shaft parallel to the tray (FIG. 4). The shaft of the handle may be attached to the meniscus tray by fasteners, welding, or other suitable means of providing secured attachment. Alternatively, the meniscus device may be formed as a single piece of material.

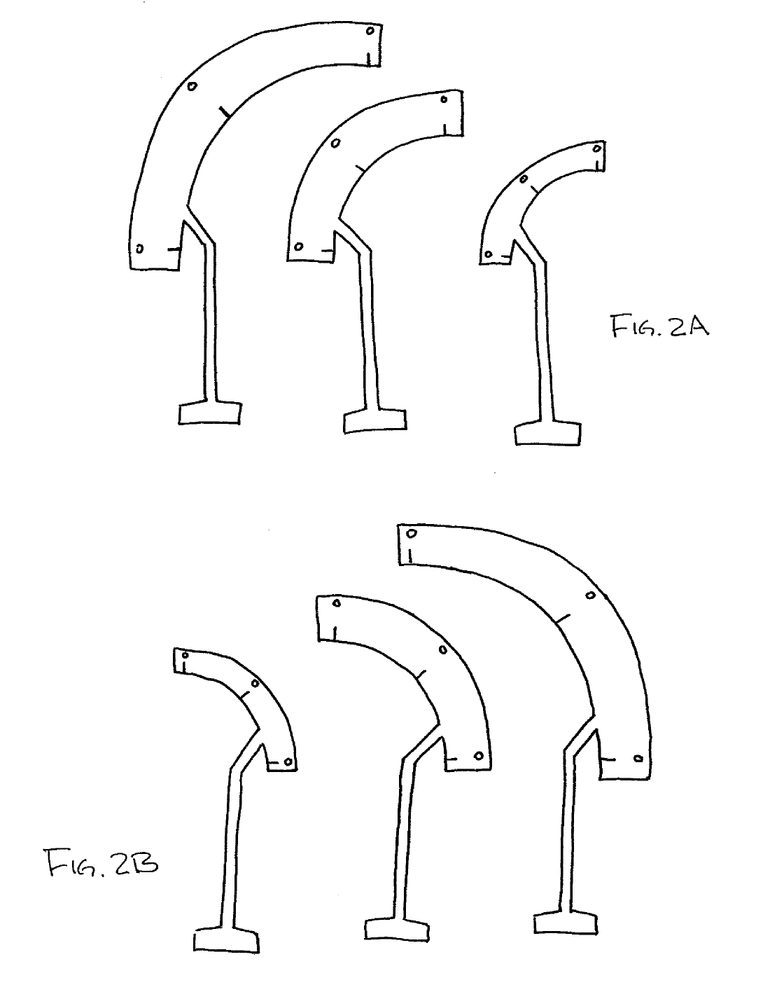

As illustrated in FIGS. 2A and 2B, meniscal trays may be provided in various sizes to accommodate different size tears of the meniscus and also various orientations. The orientations are configured as appropriate for medial and lateral meniscus sided tears on both the right knee and the left knee.

### Procedure

A human patient undergoes an arthroscopy of the knee requiring partial medial or lateral menisectomy. The surgeon determines that the amount of meniscus removed substantiates the use of collagen tissue to replace the void and improve the patient's outcome. The site is prepared by removing all poor quality meniscus, thereby creating a meniscus opening.

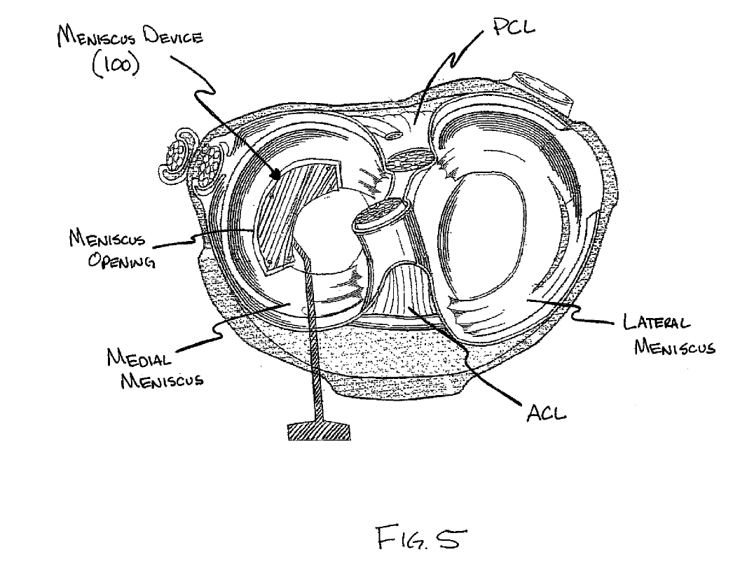

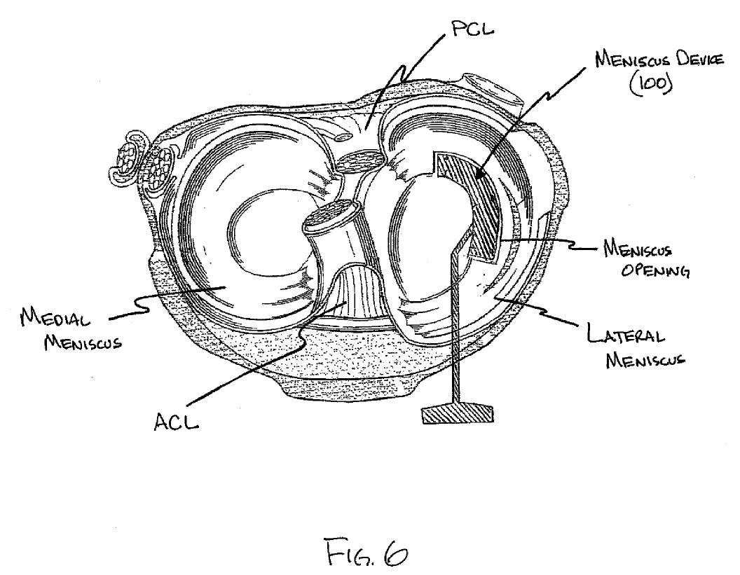

In one approach, as illustrated in FIG. 5 (and/or FIG. 6), the meniscal device is inserted into the surgical opening in the patient's knee, wherein the tray of the meniscal device is utilized to determine a proper fit and sizing of a meniscus graft within the meniscus. Medial or lateral sizers may be utilized based on the location of the pathology (FIGS. 2A, 2B, 5, 6).

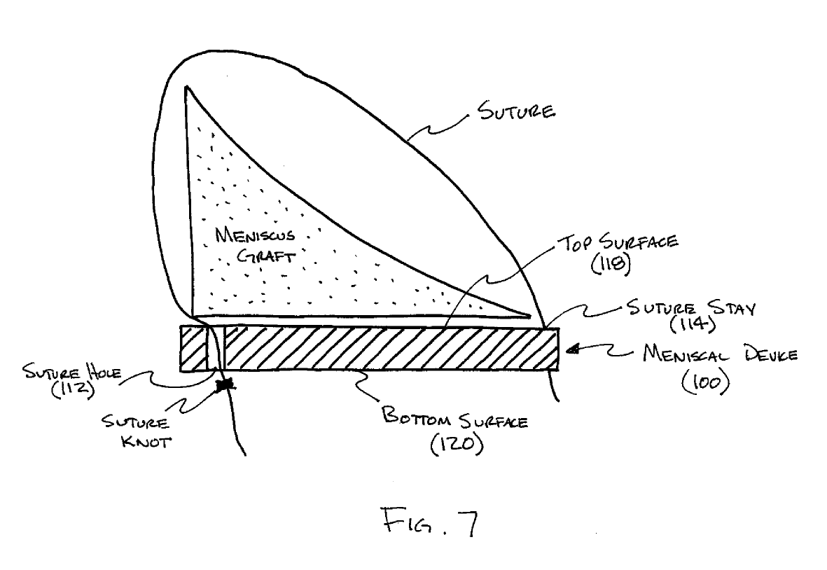

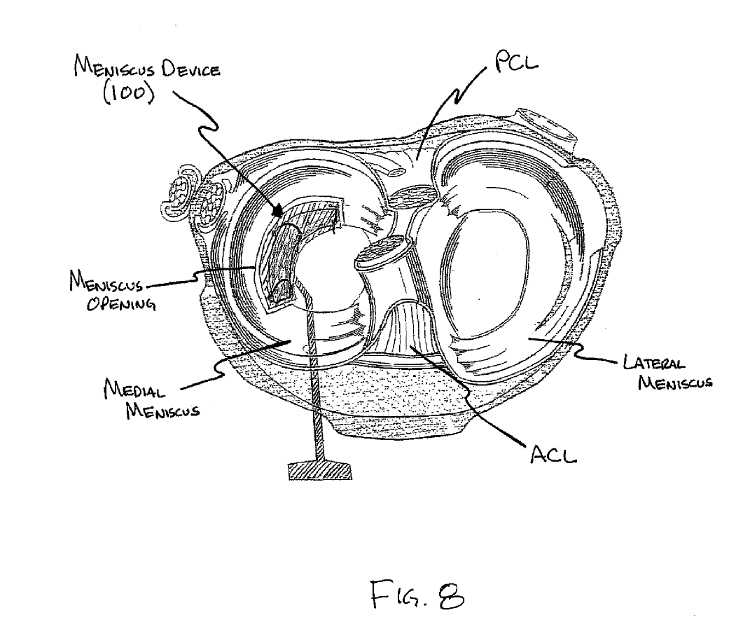

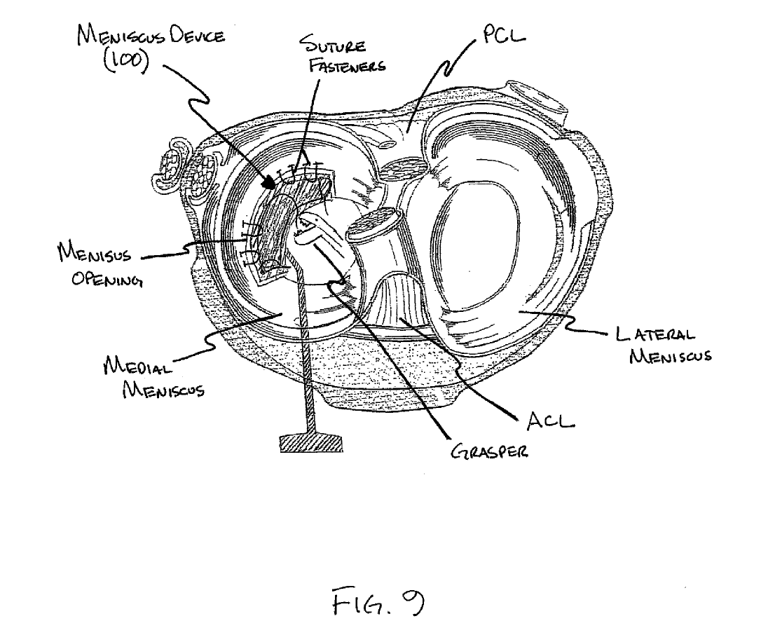

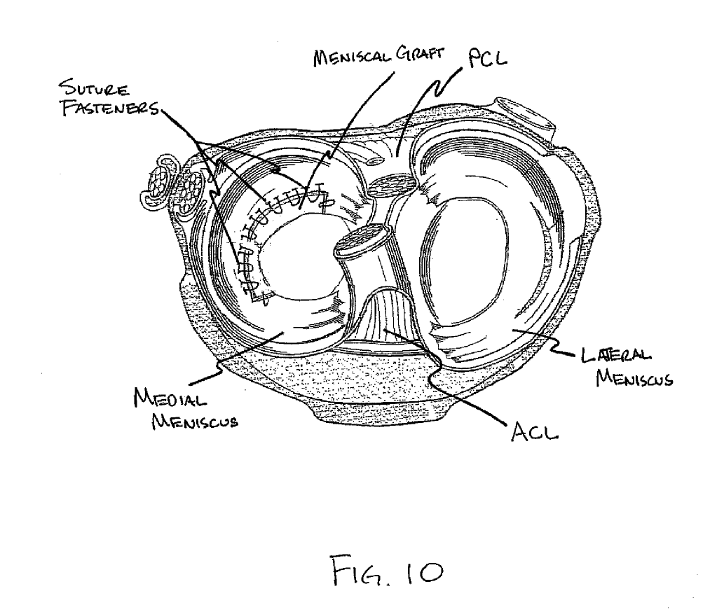

After the appropriate size of the meniscal tray is fit into the defect, the device is removed and the graft tissue is placed onto the upper surface of the tray with suture fixation (FIG. 7). A knot is tied at the bottom surface of the tray and the free end of suture is brought up through each suture hole on the outer curvature of the tray. The sutures are then brought up over the top of the graft tissue to secure it to the tray. The free end of the suture is then secured to inner curvature of the tray by placing the suture into one of the suture stays. The graft tissue is now releasably secured to the tray and may be inserted into the knee joint (FIG. 8). In one embodiment, sutures are then placed through the femoral side of the meniscal graft tissue in order to fixate the tissue to the native meniscus (FIG. 9). In one embodiment, the graft is securely attached using sutures, staples, tape, or other suitable method within the site. After the tissue is fixated to the native tissue, the sutures are released from the tray using a grasper or other suitable device (FIG. 9). The graft tissue is now attached to the native meniscus and the sizing and delivery meniscal device is now removed (FIG. 10).

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. It is therefore understood that numerous modifications may be made to the illustrative embodiments and that other arrangements may be devised without departing from the spirit and scope of the present invention.

### REFERENCE NUMERALS IN THE FIGURES ARE IDENTIFIED AS FOLLOWS:

100- meniscal device
102- tray/sizer
104- handle
106- handle end
108- inner curved surface
110- outer curved surface
112- suture hole
114- suture slit/stay
116- handle shaft
118- top surface
120- bottom surface

What is claimed is:

1. A device for sizing and implanting meniscus graft tissue, the device comprising:
a curvilinear tray having an inner curvature edge and an outer curvature edge opposite the inner curvature edge, a top surface and a bottom surface opposite the top surface, and at least one opening formed therethrough between the top surface and the bottom surface to capture at least one element configured to releasably secure the meniscus graft tissue to the curvilinear tray; and
an elongated handle extending from the curvilinear tray and including an angled shaft terminating in a handle end.

2. The device of claim 1, wherein the outer curvature edge is spaced from and substantially follows a curvature of the inner curvature edge.

3. The device of claim 1, wherein the top surface and the bottom surface of the curvilinear tray are substantially planar, and the meniscus graft tissue is configured to be releasably secured to the top surface of the curvilinear tray.

4. The device of claim 1, wherein the at least one opening of the curvilinear tray comprises at least one hole formed through the curvilinear tray proximal the outer curvature edge thereof and at least one slit formed in the curvilinear tray in the inner curvature edge thereof corresponding to and opposite of the at least one hole.

5. The device of claim 1, wherein the at least one element configured to releasably secure the meniscus graft tissue to the curvilinear tray comprises at least one suture, and the at least one opening of the curvilinear tray comprises at least one hole formed through the curvilinear tray, wherein the at least one hole is sized to accommodate the at least one suture and intercept a suture knot formed at one end of the at least one suture.

6. The device of claim 5, wherein the at least one hole is proximal the outer curvature edge of the curvilinear tray.

7. The device of claim 5, wherein the at least one hole comprises a series of holes spaced along the outer curvature edge of the curvilinear tray.

8. The device of claim 1, wherein the at least one element configured to releasably secure the meniscus graft tissue to the curvilinear tray comprises at least one suture, and the at least one opening of the curvilinear tray comprises at least one slit formed in the curvilinear tray, wherein the at least one slit is sized to provide an interference fit with the at least one suture.

9. The device of claim 8, wherein the at least one slit is formed in the inner curvature edge of the curvilinear tray and extends toward the outer curvature edge of the curvilinear tray.

10. The device of claim 8, wherein the at least one slit comprises a series of slits spaced along the inner curvature edge of the curvilinear tray.

11. The device of claim 1, wherein a majority of the shaft of the handle is in a plane spaced from and oriented substantially parallel with a plane of the curvilinear tray.

12. The device of claim 1, wherein the handle is positioned proximal one end of the curvilinear tray.

13. The device of claim 1, wherein the handle extends at a compound angle from the inner curvature edge of the curvilinear tray.

14. The device of claim 1, wherein the shaft of the handle includes a first section extending from the inner curvature edge of the curvilinear tray and a second section extending from and oriented at a first angle to the first section.

15. The device of claim 14, wherein the first section of the shaft is oriented at a second angle to the top surface and the bottom surface of the curvilinear tray, and wherein the second section of the shaft is oriented at a third angle to the first section of the shaft such that the second section of the shaft is oriented substantially parallel with the top surface and the bottom surface of the curvilinear tray.

16. The device of claim 15, wherein the first angle is in a plane substantially parallel with the top surface and the bottom surface of the curvilinear tray, and the second angle and the third angle are each in a plane substantially perpendicular to the top surface and the bottom surface of the curvilinear tray.

17. A set of meniscal devices for determining a proper fit and sizing of meniscus graft tissue, the set comprising:
at least two meniscal sizing and graft delivery devices each having a substantially planar curvilinear tray and a handle extending at a compound angle from an inner curvature edge of the curvilinear tray, the curvilinear tray including a series of holes spaced along an outer curvature edge of the curvilinear tray and a series of corresponding slits spaced along the inner curvature edge of the curvilinear tray to facilitate releasable securement of the meniscus graft tissue to the curvilinear tray,
wherein the at least two meniscal sizing and graft delivery devices have at least one of an opposing handle orientation and a different tray size.

18. The set of claim 17, wherein at least one of the at least two meniscal sizing and graft delivery devices is oriented for a lateral meniscus and at least one of the at least two meniscal sizing and graft delivery devices is oriented for a medial meniscus.

19. The set of claim 17, wherein each of the at least two meniscal sizing and graft delivery devices has a tray of a surface area in a series of different sizes in order to correspond in size and shape to meniscus graft openings of different sizes.

20. A device for implanting meniscus graft tissue, comprising:
means for appropriately sizing the meniscus graft tissue within a meniscus opening; and
means for releasably securing the meniscus graft tissue to the device while inserting and securing the meniscus graft tissue within the meniscus opening.
